(19) United States
(12) Patent Application Publication
(10) Pub. No.: US 2025/0049842 A1
(43) Pub. Date: Feb. 13, 2025

(54) THERAPEUTIC BLOOD-ACID-SHIFTING COMPOSITIONS AND METHODS

(71) Applicant: James D. Ervin, Northville, MI (US)

(72) Inventor: James D. Ervin, Northville, MI (US)

(21) Appl. No.: 18/771,555

(22) Filed: Jul. 12, 2024

Related U.S. Application Data
(60) Provisional application No. 63/526,524, filed on Jul. 13, 2023.

(51) Int. Cl. A61K 33/18; A61K 31/19; A61K 31/198; A61K 31/519; A61K 33/00; A61K 33/04; A61K 33/20; A61K 45/06

(52) U.S. Cl. CPC A61K 33/18; A61K 31/19; A61K 31/198; A61K 31/519; A61K 33/00; A61K 33/04; A61K 33/20; A61K 45/06

(57) ABSTRACT

A composition for intravenous injection into an individual's body tissue includes an acid with a pKa less than 3.0 and a conjugate base with therapeutically beneficial properties. The composition has utility in a method of shifting blood-acid for various therapeutic purposes.

### BACKGROUND

The acid-base status of the bloodstream is primarily controlled as a sum of cellular acid production in balance with renal and respiratory action to remediate blood acid. For example, physical exercise is an endeavor in which oxygen supply to cells is lacking, to induce anaerobic metabolism with corresponding lactic acid (H+, Lactate) production in cells. As acid flows from acid-producing cells to the bloodstream, the blood-acid condition temporarily shifts to become more acidic. During and after such exercise, renal and respiratory processes respond in an attempt to maintain and ultimately restore physiologic blood-acid norms. Specifically, renal processes act to extract H+ to the urine while retaining HCO₃− to the blood while carbonic anhydrase works with respiratory processes to enzymatically convert excess H+ and HCO₃− to H₂0 and CO₂, whereupon a CO₂ balance is exhaled from the lungs and H+ is stored as water. Through these processes, the bloodstream is conditioned first towards acidic, then back towards alkaline to crest slightly alkaline relative to physiologic norms in a phenomenon known as the “alkaline rebound”, before settling again back towards physiological norm. During a state of exercise-induced acid-base disturbance, oxygen status is also in flux as a consequence of increased oxygen demand in the cells, and also due to hemoglobin's reduced capacity to bear oxygen in the presence of H+, as H+ can directly displace O₂ on hemoglobin.

Given that acid-shifting and subsequent alkaline rebound is a natural and common process, it is not surprising that it is also physiologically influential. For instance, rising/falling H+ and HCO₃− gradients between the bloodstream and intracellular influence the flow of electrolytes to and from cells, falling/rising oxygen status influences various metabolic processes, falling O₂ stimulates erythropoietin for red blood cell maintenance, and various aspects of immune response, cell maintenance, growth, and healing are also triggered by changes in pH and O2 levels.

Although many therapies are aimed at increasing oxygen and reducing blood acid and related treatments, such as those described in U.S. Pat. No. 11,344,529 and U.S. Patent Pub. No. 2020/0390743 the disclosure of each of which is incorporated by reference in its entirety for all purposes, introduces use of acidic solutions to impart a bloodstream shift towards acidic as a lead element of therapy, with the anticipation that renal and respiratory compensations will subsequently restore alkaline conditions. In this way, it is proposed that elements of such therapy, termed here as “drug-induced exercise”, can induce acid-base and corresponding oxygen status changes to deliver a therapeutic benefit. Note that such stimuli are different from physiological exercise as lactate levels would not be expected to increase as a consequence of the therapy, unless lactate were specifically added to the formulation. U.S. Pat. No. 11,344,529 additionally specifically recognizes use of hydrochloric acid, ascorbic acid, dehydroascorbic acid, acetic acid, citric acid, lactic acid, phosphoric acid, and a combination of two or more thereof for administration via IV. The work further cites that various vitamins, electrolytes, antioxidants, amino acids, and other drug product can also be included for different therapeutic goals. Finally, this work introduces that use of a pH buffering agent is commonly desired to buffer the pH so-as to be compatible with infusion without causing undue irritation to veins, arteries, or other tissues. While this work represents an important first step in the art of acid-shifting therapies, the formulations as proposed do not address optimization from the standpoint of drug volume.

### SUMMARY

Disclosed herein are methods and compositions for shifting blood acid for a therapeutic purpose, while additionally presenting a therapeutic conjugate base. The methods involve administering a therapeutically effective amount of a therapeutic composition that includes an intravenous buffer solution comprising at least one pharmaceutical grade acid having a therapeutic conjugate base with a pKa<3.0, and at least one pharmaceutical grade pH buffering agent in a sterile aqueous solution. To do so, a therapeutically effective amount of a therapeutic composition may be administered that includes an intravenous buffer solution comprising at least one pharmaceutical grade acid containing at least one therapeutic conjugate base, and at least one pharmaceutical grade pH buffering agent in a sterile aqueous solution. The term “therapeutically effective amount” as used in this description and appending claims means an amount that when administered to a human or veterinary subject for treating a disease or medical condition is sufficient to effect such treatment, including delaying or preventing onset of a disease, disorder or condition, slowing or stopping progression, aggravation or deterioration of one or more symptoms of the disease, disorder or condition, ameliorating the symptoms of the disease, disorder or condition, reducing the severity of the disease, disorder or condition, and/or curing the disease, disorder or condition. The therapeutically effective amount will vary depending on the subject, the disease, disorder or condition being treated, the stage of the disease, disorder or condition being treated, the severity of the disease, disorder or condition being treated, and the manner of administration, all of which may be determined routinely by a person of ordinary skill in the art. The pharmaceutical grade acid may include Hydroiodic Acid, Perchloric Acid, Chloric Acid, Sulfuric Acid, Nitric Acid, Folic Acid, Sulfurous Acid, Sulfuric Acid, Glutamic Acid, and Pyruvic Acid, or a combination of two or more thereof. The therapeutic conjugate base may include Iodine, Perchlorate, Chlorate, Sulfate, Nitrate, Folate, Bisulfite, Glutamate, and Pyruvate, or a combination of two or more thereof, or ionized or radioactive forms thereof. The concentration of the pharmaceutical grade acid and the pharmaceutical grade pH buffering agent in the buffer solution may be sufficient to provide a total acid content of from 60 mmol/L to 3,000 mmol/L when administered to a subject. The pharmaceutical grade acid and the pharmaceutical grade pH buffering agent or agents may be selected to provide a buffer solution pH of between 1.8 and 8.6 when administered to a subject.

In some embodiments, the buffer solution is administered in an amount that is sufficient to reduce the physiological bloodstream pH of a subject by 0.01 to 1.1.

In some embodiments, the buffer solution may be administered in an amount sufficient to reduce the physiological bloodstream pH of a subject by 0.15 to 0.75.

In some embodiments, the concentration of the acid and buffering agent may provide the buffer solution with a buffer capacity sufficient to sustain the reduction of the physiological bloodstream pH of the subject for between 1 minute and 1 week.

The therapeutic intent may be treatment of diabetes, insulin resistance, glucose intolerance, hyperglycemia, hyperinsulinemia, obesity, hyperlipidemia, hyperlipoproteinemia, cancer, sepsis, trauma care, treating wounds, reducing vascular plaque, treating radiation exposure, enhancing glutathione status, enhancing sulfur-based metabolism, and/or infectious disease.

In some embodiments, the composition further comprises one or more ion sources selected from a group consisting of: a magnesium ion source, a potassium ion source, a calcium ion source, a zinc ion source, a copper ion source, a selenium ion source, a chromium ion source, a cobalt ion source, an iodine ion source, a manganese ion source, and a molybdenum ion source.

In some embodiments, the composition further comprises one or more vitamins selected from a group consisting of: a B vitamin, vitamin C, and vitamin K.

In some embodiments, the composition further comprises antioxidant defense compounds comprising one or more nonenzymatic compounds selected from the group consisting of: tocopherol (aTCP), coenzyme Q10 (Q), taurine, cytochrome c (C) and glutathione (GSH) and enzymatic components including manganese superoxide dismutase (MnSOD), catalase (Cat), glutathione peroxidase (GPX), phospholipid hydroperoxide glutathione peroxidase (PGPX), glutathione reductase (GR); peroxiredoxins (PRX3/5), glutaredoxin (GRX2), thioredoxin (TRX2), thioredoxin reductase (TRXR2), and a combination of two or more thereof.

In some embodiments, the composition further comprises one or more essential amino acids selected from a group consisting of: histidine, isoleucine, leucine, lysine, methionine, phenylalanine, threonine, tryptophan, valine and a combination of two or more thereof.

In some embodiments, the composition further comprises one or more nonessential amino acids selected from a group consisting of: tyrosine, glycine, arginine, glutamine, glutamic acid, cysteine, serine, proline, alanine, asparagine, aspartic acid, and a combination of two or more thereof.

The composition may be formulated in hypotonic, isotonic, or hypertonic form. It may be administered intravenously, by bolus, dermally, orally, optically/ophthalmically, via suppository, buccally, or via inhalation.

Administration of the compound may be performed by infusion over a period of about 1 minute to about 1 hour, and the infusion is repeated as necessary over a period of time selected from about 1 day to about 1 year.

In some embodiments, one or more alkaline-shifting solutions are employed before or after one or more acid-shifting solutions.

In some embodiments, one or more alkaline-shifting solutions are alternatively administered between one or more acid shifting solutions.

In some embodiments, one or more acid-shifting solutions are alternatively administered between one or more alkaline-shifting solutions.

The compound may be administered to a variety of subjects, including a human or veterinary subject or a culture thereof.

A pharmaceutical composition for intravenous delivery to a mammal is provided, which comprises an intravenous buffer solution comprising at least one pharmaceutical grade acid containing at least one therapeutic conjugate base; and at least one pharmaceutical grade pH buffering agent in a sterile aqueous solution.

In some embodiments, the pharmaceutical grade acid comprises an acid selected from a group consisting of Hydroiodic Acid, Perchloric Acid, Chloric Acid, Sulfuric Acid, Nitric Acid, Folic Acid, Sulfurous Acid, Sulfuric Acid, Glutamic Acid, and Pyruvic Acid, or a combination of two or more thereof.

In some embodiments, the therapeutic conjugate base comprises a base selected from a group consisting of Iodine, Perchlorate, Chlorate, Sulfate, Nitrate, Folate, Bisulfite, Glutamate, and Pyruvate, or a combination of two or more thereof, or ionized or radioactive forms thereof.

In some embodiments, the concentration of the pharmaceutical grade acid and the pharmaceutical grade pH buffering agent in the buffer solution is sufficient to provide a total acid content of from 60 mmol/L to 3,000 mmol/L when administered; and wherein the selected pharmaceutical grade acid and the pharmaceutical grade pH buffering agent or agents provide a buffer solution pH of between 1.8 and 8.6 when administered.

### BRIEF DESCRIPTION OF THE DRAWINGS

FIG. 1 shows an illustration of acid shifting that may result from embodiments disclosed herein.

FIG. 2 shows solutions for therapeutic use at a target osmolarity according to embodiments disclosed herein. A hypo-osmolar solution has a lower osmolarity (concentration of solutes) and hyper-osmolar solution has a higher osmolarity compared to a target solution.

FIG. 3 shows examples of using acid shifting while targeting indications served by inclusion of therapeutic bases according to embodiments disclosed herein. Package efficiency at a target osmolarity is formulated with acids that contain the therapeutic base as opposed to excipients paired with the acid and/or base.

FIG. 4 shows an illustration of the total titratable acid (mol H+/L) in a given volume at target osmolarity. It can be seen that the total is greater when formulated with acids that contain the therapeutic base as opposed to excipients paired with the acid and/or base.

FIG. 5 shows examples of acid-shifting solutions and associated calculations according to embodiments disclosed herein.

### DETAILED DESCRIPTION

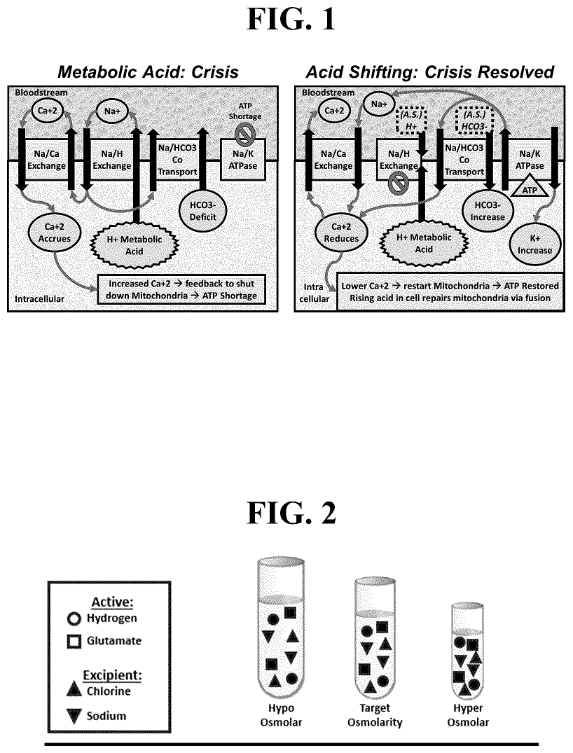

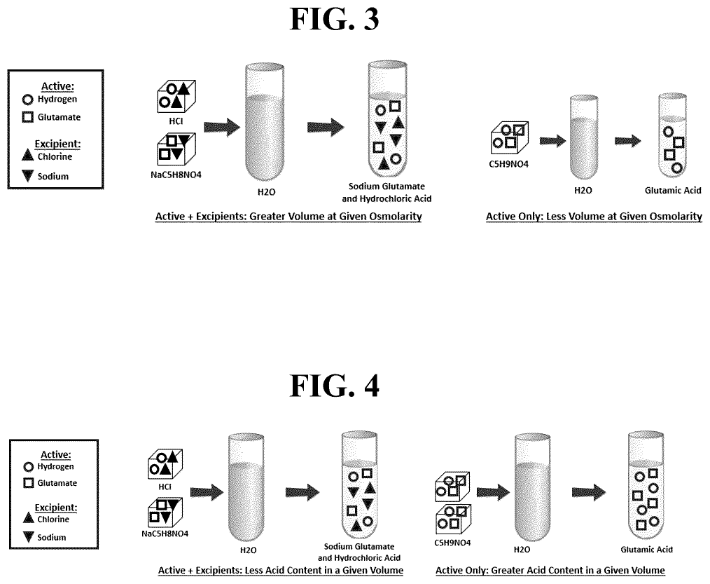

As illustrated by FIG. 1, it is believed that acid shifting in the bloodstream acutely impairs acid extrusion from the cell to facilitate a recovery of the intracellular metabolism. This is relevant because in the case of chronic metabolic acid production and acid extrusion from the cell, the net exchange of ions can reduce intracellular buffer stores while increasing intracellular calcium. As intracellular calcium increases, it creates a feedback that selectively disables the electron transport chain (ETC) to reduce oxidative phosphorylation and net adenosine triphosphate (ATP) yield. Reduced ATP additionally impairs the action of the Sarcoendoplasmic Reticulum Calcium ATPase (SERCA), which function to regulate intracellular Ca+2 by concentrating it in the reticulum reservoirs. Finally, reduced ETC activity also reduces the progression of H+ to H2O, which further increases metabolic acid production.

If acid is introduced into the bloodstream, for example via an acid shifting drug according to embodiments disclosed herein, the sodium hydrogen exchanger becomes stalled to directly reduce sodium intake and consequently reduces calcium intake through the Na/Ca exchanger. Intracellular hydrogen levels may rise while the efflux of H+ is impaired, which may increase the chemiosmotic gradient and acutely increase available ATP. More acidic intracellular conditions are also recognized to stimulate cardiolipin remodeling and mitochondrial fusion and repair processes. Higher ATP levels also may increase the action of the Na/K ATPase to import potassium while increasing bloodstream sodium levels to stimulate the HCO3/Na co-transport to import HCO3− buffers. The bloodstream sodium can then act through the sodium calcium exchanger to reduce intracellular calcium. Elevated ATP may additionally restore action of the SERCA, which in turn reduces intracellular Ca+2 by concentrating it in the reticulum reservoirs. Finally, the falling Ca+2 concentrations may reduce feedback to authorize restoration of ETC activity to promote H+ to H2O while increasing metabolic yield.

Oxygen delivery is also expected to be acutely affected as a result of this process. As blood acid increases, the efficiency of hemoglobin to transport O2 is reduced as H+ competes for these same sites. Consequently, O2 delivery may be acutely reduced. Although ATP production through oxidative processes might be reduced, the rise in blood acid and corresponding intracellular acid increase would be expected to increase the chemiosmotic gradient to increase ATP from oxidative phosphorylation. Concurrently, the reduction in O2 based-metabolism would be expected to reduce oxidative stress and net Reactive Oxygen Species (ROS) levels, where reduced ROS would be expected to further provide feedback to restore ETC activity to increase yield of future metabolism towards more ATP with more metabolic H+ completing the conversion into H2O through the ETC. This would consequently reduce metabolic acid efflux from the cell. Following infusion, renal and respiratory compensations would be expected to achieve an alkaline rebound, which would enhance O2 delivery as there would be fewer H+ competing with O2 for sites on hemoglobin. This process of restoring ETC function by reducing ROS through reduced O2 servicing has been recognized in research as a potential mechanism to reduce reperfusion injury following hypoxia. These are the proposed underpinnings of acid shifting therapeutics.

Disclosed formulations for intravenous or intra-arterial infusion, or other means of delivery within a subject minimize package volume or infused volume. Regarding therapeutic compositions for drug-induced exercise involving acid shifting, acids that have a low pKa (or pKa1 or pKa2), for example below pH 3.0, would be especially valued as they offer concentrated means to package H+ compared to weaker acids. Furthermore, it is recognized that several acids with low pKa also have conjugate bases with therapeutically useful properties. For example, the following compounds are recognized to have therapeutic value as conjugate bases and can exist as acids and exhibit one or more pKa<3.0: Iodine, Perchlorate, Chlorate, Sulfate, Nitrate, Folate, Bisulfite, Glutamate, and Pyruvate per the table below:

TABLE 1
Therapeutic Conjugate Bases and Associated Acids with pKa < 3.0

| Conjugate Base | Acid | HA | A− | pKa |
|---|---|---|---|---|
| Iodine | Hydroiodic | HI | I− | (Ka > 1, pKa < 1). |
| Perchlorate | Perchloric | HClO4 | ClO4− | Strong acids |
| Chlorate | Chloric | HClO3 | ClO3− | completely |
| Sulfate (1) | Sulfuric (1) | H2SO4 | HSO4− | dissociate in |
| Nitrate | Nitric | HNO3 | NO3− | aq solution |
| Folate (1) | Folic (1) | C19H19N7O6 | C19H18N7O6− | 1.63 |
| Bisulfite (1) | Sulfurous (1) | H2SO3 | HSO3− | 1.81 |
| Sulfate (2) | Sulfuric (2) | HSO4 | SO4−2 | 1.92 |
| Glutamate (1) | Glutamic (1) | C5H9NO4 | C5H8NO4− | 2.19 |
| Pyruvate | Pyruvic | C3H4O3 | C3H3O3− | 2.49 |
| Folate (2) | Folic (2) | C19H19N7O6− | C19H18N7O6−2 | 2.71 |

While acid-shifting infusible drugs can be compounded by combining a balance of salts containing a therapeutic base to a composition utilizing an acid and an excipient base, such a composition would require added dilution, hence volume, to realize a given osmolarity target. Such an approach would increase both the package size and infused volume of the drug product. An acid choice to realize a target osmolarity while maintaining a minimum package volume or infused volume can be an acid that itself contains the therapeutic base. For example, if glutamate administration is desired concurrent with acid shifting, then glutamic acid would achieve minimum infused volume at a given osmolarity compared to a composition comprised of a base-excipient acid, like HCl, and a secondary complement of glutamate salts. If a target molar dose of glutamate were less than a molar H+ (pH) target, then a suitable base-excipient acid could be utilized alongside a balance of glutamic acid at pKa(1) pH level to realize the final H+ and glutamate targets at minimum volume. If a target molar dose of glutamate were equal to a molar H+ (pH) target, then a balance of glutamic acid at pKa(1) pH level alone could realize both H+ and glutamate targets at minimum volume. If a target molar dose of glutamate were greater than a molar H+ (pH) target, then a balance of glutamic acid at pKa(1) pH level could be combined with a glutamate salt to realize the final H+ and glutamate targets at minimum volume.

As further context on drug design utilizing pKa, the pKa value is the pH at which half of the acid molecules are protonated and half are deprotonated. When the pH is below the pKa value, a higher fraction of acid molecules are protonated. Alternately, when the pH is below the pKa value, a higher fraction of acid molecules are deprotonated. For example, if an acid has a pKa of 4.5 and the pH of the solution is 2, then most of the acid molecules will be protonated (HA) and very few will be deprotonated (A−). Thus, utilizing acids in forms above or below their pKa is an alternative means to adjust the ratio of H+ and A− that present in the drug product. Equilibrium mechanisms can be considered to ensure that acid species do not convert to other forms, such as carbonic acid, which could convert H+ to H2O and reduce the net presentation of H+.

Examples of acid-shifting choices having pKa<3.0 are presented below for select therapeutic conjugate bases, along with recognized potential applications of interest:

Common Iodine (Hydroiodic Acid, Strong Acid, pKa1<1)
a. Support thyroid hormone production to overcome hypothyroidism (not appropriate for hyperthyroidism)
b. Treat and prevent some goiters. i.e. enlarged thyroid gland.
c. Neurodevelopment during pregnancy to support brain development in fetuses.
d. Neurodevelopment during nursing to support brain development in babies.
e. Neurodevelopment during childhood to support brain development.
f. Treat fibrocystic breast disease, a non-cancerous condition most common in women of reproductive age, marked by painful breast lumps.
g. Protection from nuclear fallout where iodide protects the thyroid gland that might otherwise be susceptible to radiation injuries.

Perchlorate (Perchloric Acid, pKa<1)
h. Perchlorate is a potent inhibitor of osteoclast function and acts through an influence on intracellular [Ca2+], and in turn upon the degree of cell retraction in osteoporosis.

Chlorate (Chloric Acid, pKa<1)
i. Chlorate infusion reduces the fecal shedding of Escherichia coli.

Radioactive Iodine (Hydroiodic Acid, Strong Acid, pKa1<1)
j. Manage overactive thyroid gland via use of radioactive iodine, to destroy extra thyroid cells.
k. Treatment option for thyroid cancer to destroy thyroid cancer cells.

Sulfate (Sulfuric Acid, Strong Acid, pKa1<1, pKa2=1.92) and/or Bisulfite (Sulfurous Acid, pKa1=1.18)
l. Sulfate and Sulfite which oxidizes to sulfate to promote synthesis of sulfur-containing amino acids (SAAs) such as methionine, cysteine, cystine, homocysteine, homocysteine, and taurine with specific potential applications for vegans, athletes, children, or patients with HIV, because of an increased risk for SAA deficiency.
m. Promote SAA synthesis of secondary sulfur-containing compounds in the body: S-adenosylmethionine (SAMe), glutathione (GSH), N-acetylcysteine (NAC), dimethyl sulfoxide (DMSO), glucosamine and chondroitin sulfate, which may have clinical applications in the treatment of several conditions such as depression, fibromyalgia, arthritis, interstitial cystitis, athletic injuries, congestive heart failure, diabetes, cancer, and AIDS.

Nitrate (Nitric Acid, Strong Acid, pKa<1)
n. Nitrate for conversion to NO via low pH or action of the enzyme xanthine oxidoreductase under low oxygen conditions, like ischemia.
o. Nitrates as Diuretics to enhance excretion of urinary chloride and sodium, resulting in a net loss of salt and water caused by increased glomerular filtration without an equivalent increase in tubular reabsorption.

Folate (Folic Acid, pKa1=1.63, pKa2=2.71)
p. To treat or prevent certain anemias by promoting red blood cell growth caused by poor diet, pregnancy, alcoholism, liver disease, certain stomach/intestinal problems, kidney dialysis, or other conditions.
q. Prevent spinal cord birth defects by promoting proper cell growth.

Glutamate (Glutamic Acid, pKa=2.19)
r. Promote learning and memory by interacting with four different receptors so messages are successfully and quickly sent between nerve cells.
s. Serve as an energy source for brain cells when glucose levels are low, such as during hypoglycemic conditions.
t. Sleep-wake cycle influence as glutamate levels are high when awake and also during the rapid eye movement (REM) phase of sleep.
u. Provide pools of glutamic acid to work with stores of cysteine and glycine to restore glutathione; for reduction of oxidative stress, maintaining redox balance, enhancing metabolic detoxification, and regulating the immune system. Various chronic, age-related diseases such as those related to neurodegeneration, mitochondrial dysfunction, and even cancer, have been related to suboptimal or deficient glutathione levels.

Pyruvate (Pyruvic Acid, pKa=2.49)
v. Pyruvate directly supports pyruvate impaired central carbon metabolism and serves as an important H₂O₂ scavenger for Lyme disease/pathogenic spirochetes.
w. Pyruvate supplementation as an ergonomic aid to enhance work output.
x. Pyruvate supplementation to promote weight loss or reduce circulating glucose by providing energy that bypasses the ability of the body to store glucose as body fat.

Beyond delivering acid shifting in the presence of a therapeutically beneficial conjugate base, compositions may also benefit from the presence of components that address other needs or deficiencies. For instance, therapeutic acid-shifting compositions may be further enhanced by addition of vitamins, mineral electrolytes, antioxidants, amino acids, pain management drugs, or other drug products, including B vitamins, vitamin C, and/or vitamin K.

Beyond addition of components for therapeutic purposes, it is also anticipated that potential components may be included to participate as a conjugate base or “buffer”. Specifically, such buffer components would target to support as-administered adjustment of pH into a specific pH range to impart an acid-shifting stimulus in the presence of other therapeutically beneficial components in a manner that is physiologically compatible with tissues. For instance, the ideal pH range for intravenous care is commonly expressed between 6.5 and 8.0, to avoid irritation in veins and arteries. In intramuscular injections, a pH range between 2 and 11 is acknowledged while in subcutaneous injections, the range may lie between 4 and 9. In dermal applications, topical creams are commonly formulated in a pH range between 4 and 6 recognizing that the natural “acid mantle” of the skin is between 4.4 and 6.0. In other tissue compartments, such as the vagina, a lower pH may be appropriate as the common pH of vaginal secretions is between 3.8 and 5.0. In the pancreas, pancreatic juice has a pH between 7.8 and 8.8. Thus, acid shifting formulations may be designed to target various pH “as administered” across a range below 2.0, as in muscles, and below 8.8, as in the pancreas. Thus, a pH range of 1.8 to 8.6 is anticipated as potentially useful for the purposes of acid shifting. In each application, components may be optimized based on their ability to buffer and improve acid stability, e.g. quasi-equilibrium, during the administration process.

For purposes of enhancing stability of compositions at or near a specific pH for administration, many choices of buffering compounds may be utilized, where each may be more or less suitable at a given pH. For instance, in the pH range from 1.8 to 3.0, buffers, like Phosphate, may be used independently or alongside therapeutic conjugate buffers like Folate, Bisulfite, Sulfate, Glutamate, or Pyruvate. In the pH range from 3.0 to 5.0 pH, conventional buffers like Citrate, Lactate, Ascorbate, or Acetate may be used independently or alongside therapeutic conjugate buffers such as Nitrite, Glutamate, or Folate. In the pH range of 5.0 to 7.0, conventional buffers like Citrate, Acetate, or Bicarbonate may be used independently or alongside therapeutic conjugate buffers such as Folate or Bisulfite. In the pH range above 7.0, conventional buffers like Phosphate and Ascorbate may be used independently or alongside therapeutic conjugate buffers such as Glutamate. Thus, many alternative buffer choices can be considered to achieve a target pH administration, including those from the set of so-called Therapeutic Conjugate Bases for Acid Shifting/Therapeutic Buffers, as listed in table 2. Such therapeutic buffer choices may be desired whenever the solution calls for a greater fraction of therapeutic base relative to the proton (H+) fraction and when the pKa of the buffer is near to the pH target as administered.

TABLE 2
Buffer Capacity of Recognized Therapeutic Conjugate Bases as Desired
For Acid Shifting Within 1.8 to 11.5 pH Target Administration Window

| Conjugate Base | Acid | HA | A− | pKa |
|---|---|---|---|---|
| Folate (1) | Folic (1) | C19H19N7O6 | C19H18N7O6− | 1.63 |
| Bisulfite (1) | Sulfurous (1) | H2SO3 | HSO3− | 1.81 |
| Sulfate (2) | Sulfuric (2) | HSO4 | SO4−2 | 1.92 |
| Glutamate (1) | Glutamic (1) | C5H9NO4 | C5H8NO4− | 2.19 |
| Pyruvate | Pyruvic | C3H4O3 | C3H3O3− | 2.49 |
| Folate (2) | Folic (2) | C19H19N7O6− | C19H18N7O6−2 | 2.71 |
| Nitrite | Nitrous | HNO2 | NO2 | 3.39 |
| Glutamate (2) | Glutamic Acid (2) | C5H9NO4− | C5H8NO4−2 | 4.25 |
| Folate (3) | Folic Acid (3) | C19H19N7O6−2 | C19H18N7O6−3 | 4.68 |
| Bisulfite (2) | Sulfurous (2) | HSO4− | SO4−2 | 6.91 |
| Folate (4) | Folic Acid (4) | C19H19N7O6−3 | C19H18N7O6−4 | 7.53 |
| Glutamate (3) | Glutamic Acid (3) | C5H9NO4−2 | C5H8NO4−3 | 9.67 |

In contrast to pH, which measures the concentration of free protons in a solution, titratable acidity measures the sum of free protons and un-dissociated acids in a solution. For definition purposes, titratable acidity is defined here as the sum of free protons and un-dissociated acids in a solution between a pH as administered and a final pH of 7.4. For a specific formulation to accomplish acid shifting, it should contain a total titratable acid content that is suitable for the therapeutic goal of shifting acid levels relative to adjacent compartments, tissues, cells, or organelles. To distinguish from incidental inclusion of acids in a drug product not designed for acid-shifting, a level greater than 60 mmol/L has been defined previously, such as in U.S. Pat. No. 11,344,529. This level distinguishes “incidental presence of acids” in a drug product from purposeful inclusion of acids for the purpose of effecting an acid shifting effect. In essence, formulating with the intent to deliver a high-titratable acid content requires additional considerations as discussed in the context of buffering to meet pH targets as administered. For this reason, drug designs are not expected to exceed 60 mmol/L titratable acidity unless acid shifting were intentionally a part the drug design.

The total amount of acid introduced into the bloodstream and the fraction extracted through renal and respiratory processes can vary depending on individual factors such as diet, metabolism, and overall health. On average, the human body produces about 50 to 100 milliequivalents (mEq) of acid per day as primarily generated as a result of metabolic processes. The renal system, specifically the kidneys, extracts around 80% of the acid as produced into 800 ml to 2000 ml of urine per day. The 20% balance of acid is removed by the respiratory system, mainly through the exhalation of carbon dioxide (CO2), which works with water (H2O, H+ bound to 0-2) to maintain equilibrium with carbonic acid (H2CO3) in the bloodstream.

The titratable acidity of urine can be defined as the amount of acid eliminated per liter of urine or alternately as the amount of alkali required to adjust the pH level of urine back to the pH level of the original blood plasma, which is ˜7.4 in mammalian species. As such, the titratable acidity of typical mammalian urine varies from ˜20 to 50 mmol/1 under normal conditions, as high as 60 mmol/1 upon ingestion of acid-forming food, and as high as 80 mmol/l. during fasting. Thus, therapeutic compositions utilizing 60 mmol/L of titratable acid content contain as much or more acid than common urine. Moreover, an infusion of 500 ml of a 60 mmol/L solution would be equivalent to infusing roughly a quarter of a day of acid load as rejected through renal and respiratory processes. Thus, even solutions with a minimum of 60 mmol/L of titratable acidity can be expected to be physiologically influential.

The upper bound on total titratable acid content is limited only by the concentration potential of an acid itself. For instance, considering a formulation based solely on a strong acid, such as hydroiodic acid at 1 M (mol/L) or pH=0, the initial titratable acid content is 1 M or 1,000 mmol/L since hydroiodic acid is fully ionized at this pH. Because the pKa value for hydroiodic acid is <1 (˜−9.4), the ratio of [I−]/[HI] is ˜1 at a physiologic pH of 7.4, resulting in a final titratable acid content of ˜1 mmol/L. Therefore, the change in, i.e. total, titratable acid content would be 999 mmol/L. Recognizing this, 3,000 mmol/L is suggested as a practical upper bound for purposes of parameterizing description.

Administered formulations can be lower in pH than that of a target compartment, whether the target is the bloodstream, the interstitial space, the intracellular, or another. There may also be value to precede or follow-up an acid-shifting administration with one that is alkaline relative to the compartment of interest, so-as to chemically force an “alkaline displacement” or “alkaline rebound”. Acid-alkaline oscillation could also be induced by alternative administration of acid shifting and alkaline shifting solutions relative to compartments of interest. Given that ion exchange processes between the bloodstream and intracellular, interstitial and intracellular, and intracellular and organelles often have directionally defined frequency response characteristics, principles of resonance might be exploited to condition specific damping or exaggerated response regarding induced ion exchange.

Acids with a pKa less than 3.0 whose conjugate base offers therapeutically beneficial properties, “therapeutic acid”, reduce package volume and infused volume of therapeutic acid-shifting compositions. Compositions can be developed to target molar acid quantity less than, equal to, or greater than the target molar quantity of the conjugate base. Additionally, multiple acid having pKa<3.0 with therapeutically beneficial conjugate bases are utilized together. These cases are introduced below as additionally affected through use of acids at pH above and below relevant pKa. After compounding to achieve a target molar ratio of components, solutions may be diluted to achieve a target osmolarity that is hypotonic (human ref.: <270 Osm/L), isotonic (human ref.: 270-310 Osm/L) or modified with excipients like NaCl to make the composition more hypertonic (human ref.: >310 Osm/L).

Compositions involving single therapeutic acids can be readily designed using the Henderson Hasselbach equation, which relates the pH of a solution to the ratio of the molar quantity of a conjugate base to the acid in context of its pKa:

$$pH = pKa + log ( conjugate base / acid )$$

An acid not containing a target therapeutic conjugate base, such as Hydrochloric acid, may be formulated at a pH of 2.19 and combined with an additional molar balance of a glutamate salt, such as sodium glutamate to achieve the target ratio of glutamate to H+. Such a solution would require additional dilution, hence volume, relative to a solution which utilized a therapeutic acid in some part.

When the target molar acid is less than the target molar quantity of the conjugate base, a balance of therapeutic acid may be formulated to have a pH above the relevant pKa and/or must be combined with a balance of therapeutic salt. For example:

A therapeutic acid-shifting composition having 1 mol H+ per 2 mol Glutamate (pKa=2.19), could be formulated at a target pH of:

$$pH = pKa + log ( conjugate base / acid ) = 2.19 + log ( 2 / 1 ) = 2.49$$

Alternately as motivated by material availability, glutamic acid could be formulated at a pH of 2.19, and an additional molar quantity of a glutamate salt, such as sodium glutamate could be added to increase the ratio of glutamate without increasing H+.

Alternately for the purpose of contrast, an acid not containing the target therapeutic conjugate base, such as Hydrochloric acid, could be formulated at a pH of 2.49 and combined with an additional molar balance of a glutamate salt, such as sodium glutamate to achieve the target ratio of glutamate to H+. Such a solution would require additional dilution, hence volume, relative to a solution which utilized a therapeutic acid in some part.

When the target molar acid quantity is equal to the target molar quantity of the conjugate base, a balance of therapeutic acid may be formulated to have a pH at the relevant pKa.

A therapeutic acid-shifting composition having 1 mol H+ per 1 mol Glutamate (pKa=2.19) could be formulated at a target pH of:

$$pH = pKa + log ( conjugate base / acid ) = 2.19 + log ( 1 / 1 ) = 2.19$$

Alternately as motivated by material availability, glutamic acid could be formulated at a pH below 2.19, while an additional molar quantity of a glutamate salt, such as sodium glutamate could be added to increase the ratio of glutamate to equal the net H+ quantity.

Alternately for the purpose of contrast, an acid not containing the target therapeutic conjugate base, such as Hydrochloric acid, could be formulated at a pH of 2.19 and combined with an additional molar balance of a glutamate salt, such as sodium glutamate to achieve the target ratio of glutamate to H+. Such a solution would require additional dilution, hence volume, relative to a solution which utilized a therapeutic acid in some part.

When the target molar acid quantity is greater than the target molar quantity of the conjugate base, a balance of therapeutic acid may be formulated to have a pH below the relevant pKa. A therapeutic acid-shifting composition having 2 mol H+ per 1 mol Glutamate (pKa=2.19), could be formulated at a target pH of:

$$pH = pKa + log ( conjugate base / acid ) = 2.19 + log ( 1 / 2 ) = 1.89$$

Alternately as motivated by material availability, glutamic acid could be formulated at a higher pH, such as at pKa=2.19, while an additional molar quantity of a conjugate excipient acid, such as HCl could be added to increase the ratio of H+relative to glutamate.

Alternately for the purpose of contrast, an acid not containing the target therapeutic conjugate base, such as Hydrochloric acid, could be formulated at a pH of 1.89 and combined with an additional molar balance of a glutamate salt, such as sodium glutamate to achieve the target ratio of glutamate to H+. Such a solution would require additional dilution, hence volume, relative to a solution which utilized a therapeutic acid in some part.

A composition involving multiple therapeutic conjugate bases has a total target molar acid quantity that is equal to the sum of equal parts of two conjugate bases. In this case, two therapeutic acids may be combined in a ratio appropriate manner as formulated to each have a pH at their relevant pKa.

A therapeutic acid-shifting composition having 2 mol H+ per 1 mol Glutamate (pKa=2.19) and 1 mol of Pyruvate (pKa=2.49) could be formulated at target pH levels of 2.19 and 2.49 respectively. Then the concentration of H+ (at pKa this equals the conjugate base concentration) could be calculated for each acid so that an appropriate mixing ratio could be identified to yield the target result. This can be readily determined by utilizing the standard relation between pH and concentration:

$$[ H + ] = {10}^{- pH}$$

Thus, the target ratio of mixing would be:

$${[ H + ]}^{GA} / {[ H + ]}^{PA} = {10}^{- 2.49} / {10}^{- 2.19} = 0.00645 / 0.00323 = 2.$$

Because the concentration of Glutamic Acid is double that of Pyruvic Acid when each is compounded at pH=pKa, two parts of Pyruvic Acid would need to be mixed per each part Glutamic acid to realize a 2:1:1 formulation of H+:Glutamate:Pyruvate.

Alternately as motivated by material availability, Glutamic Acid and or Pyruvic Acid could be formulated at pH levels below, at, or above their respective pKa values. In such cases, additional molar quantities of glutamate salts, pyruvate salts, or acids with excipient conjugate bases may be required to realize a 2:1:1 formulation of H+:Glutamate:Pyruvate, with an additional excipient concentration present. The presence of a small fraction of excipients in such a case would require a modest level of additional dilution to realize a target osmolarity.

Alternately for the purpose of contrast, an acid not containing the target therapeutic conjugate base targets, such as Hydrochloric acid, could be combined with an additional molar balance of a glutamate and pyruvate salts, but would require additional dilution, hence volume, relative to a solution which utilized one or more therapeutic acids in some part. Such an approach may require up to two times more dilution, hence volume, than a solution which utilized at least one therapeutic acid in part.

In some embodiments disclosed herein, it may be preferred for a selected pharmaceutical grade acid and/or the buffering agent(s) to provide a buffer solution pH of between 1.8 and 8.6 when administered to a subject. Generally, a pH target of interest for therapeutic administration of acidic solutions should be chosen with regard to the pH tolerance of the surrounding tissue and the gradient effect to be imparted. After identifying a pH of interest, the methodology to stabilize acids at a target pH is well established. It involves selecting a buffer that is physiologically tolerable and/or beneficial and whose pKa is near in value to the desired pH as administered. In simple systems involving a single buffer, the Henderson-Hasselbalch equation can be rearranged to define the target ratio of the quantity of base to acid with quantity expressed in units such as M or mM:

$$base / acid = 10 \hat{} ( pH - pKa )$$

As an example, a solution targeting a final pH of 1.8 as disclosed herein may use a glutamate buffer, with a pKa of 2.2. Glutamate is physiologically tolerable and further recognized as a therapeutic agent. If a target acid concentration and therapeutic volume were defined, then the appropriate glutamate buffer amount could be defined as demonstrated Table 3.

Similarly, a solution targeting a final pH of 8.6 may utilize a carbonate buffer, as its pKa is 10.3 and it is physiologically tolerable. If a target acid concentration and therapeutic volume were defined, Then the appropriate carbonate buffer amount could be defined as demonstrated in Table 3.

TABLE 3
Buffer choice example to stabilize 0.5 M/L
Sulfuric Acid at 1.8 pH

| | | Buffer | Buffer | | Acid | Acid | | |
|---|---|---|---|---|---|---|---|---|
| | Volume | concentration | amount | | concentration | amount | H+ free at | Glutamate |
| | (ml) | (M/L) | (mM) | pH | (M/L) | (mM) | pH (M) | pKa |
| AS | 10 | 0.2037 | 2.04 | 1.80 | 0.5000 | 5.0 | 1.58E−02 | 2.2 |
| Solution | | | | | | | | |

Buffer choice example to stabilize 0.5 M/L
Sulfuric Acid at 8.6 pH

| | | Buffer | Buffer | | Acid | Acid | | |
|---|---|---|---|---|---|---|---|---|
| | Volume | concentration | amount | | concentration | amount | H+ free at | Carbonate |
| | (ml) | (M/L) | (mM) | pH | (M/L) | (mM) | pH (M) | pKa |
| AS | 10 | 0.0100 | 0.10 | 8.60 | 0.5000 | 5.0 | 2.51E−09 | 10.3 |
| Solution | | | | | | | | |

In some embodiments, the buffer solution may be administered in an amount sufficient to reduce the physiological bloodstream pH of the subject by 0.01 to 1.1. The Henderson-Hasselbalch relationship also may be used to design compositions to achieve various pH shifting results. In practice, the degree of blood shifting depends on the volume of acid shifting drug administered, the concentration of acid and buffer components in the drug and also in the blood, dilution from concurrent sources like saline, the rate of administration, and the degree of renal and respiratory response elicited in the patient. If the drug delivery is fast relative to renal and respiratory compensation processes, say as measured in seconds or minutes, then a corresponding blood pH shift can be expected.

In simplified terms, buffers are bases that stabilize hydrogens (acids) by associating with them at a given pH. Buffers or bases are chosen for a given application on the basis of their pKa, or acid dissociation constant. This affects the strength of an acid when in solution with the buffer. Specifically, the pKa represents the pH at which half of the acid molecules are dissociated into their conjugate base and a hydrogen ion (H+). In other words, pKa indicates the tendency of an acid to donate a proton (H+) in aqueous solution.

In the interest of shelf life, an acidic drug may need to be stored for an extended period. Although it is commonly desired that solutions be administered with pH>6.0 to avoid vein irritation, acids in this range must exist in equilibrium with H₂O and CO₂ though the carbonic acid form, which has a pKa of 6.4.

$$CO 2 + H 2 O ⇌ {H}_{+} + HCO 3 -$$

This means that packaging of acids near pH=6.4 requires handling of gas-phase pressures. As an alternative, a drug may be compounded in two parts. One part may utilize a low pKa buffer, such as a therapeutic base having a pKa<3, and formulated at a low pH, e.g. 2-3. Such an arrangement allows for the drug part to be shelf stable without substantial gas-phase interactions. Then, prior to administration, it may be mixed with a second part comprising a buffer, such as bicarbonate, to achieve a pH>6.0 for administration. Although this “as-mixed” solution would also be subject to carbonic acid instability, the incidental off-gassing of CO2 may be minimized during administration through use of closed vessels and by shortening the time between mixing and use.

Recognizing these elements, the total acid content in an acid-shifting solution required to impart an acid shifting effect in blood as disclosed herein may be calculated using knowledge of acids and buffers. Speaking first to the blood and plasma, the primary buffers are:
1. Bicarbonate (HCO3−): This is the most significant buffer in the blood and is responsible for approximately 60-70% of the blood's buffering capacity.
2. Proteins: Hemoglobin in red blood cells account for approximately 20-30% of blood buffering while other plasma proteins account for another 5-7%
3. Phosphate Buffers: Comprise about 1-3% of bloodstream buffering, so they play a minor role in the blood but are more significant in the renal system.
4. Organic Acids: Such as lactic acid, which can vary with metabolic activity, can also play a role.

For the sake of illustration, it may be presumed that bicarbonate contributes 65% towards the total buffer capacity and, further, that the total effective buffer capacity in the blood can be approximated by scaling the amount of bicarbonate by 100%/65%. The average concentration of bicarbonate (HCO3−) in the bloodstream typically ranges from 22 to 29 millimoles per liter (mmol/L) and the average blood volume for an adult human is about 5 liters, though this can vary based on several factors such as age, sex, body size, and overall health. Considering conditions where administration into a patient is conducted relatively quickly, so that renal and respiratory compensations may be ignored, a net blood-shifting effect can be estimated.

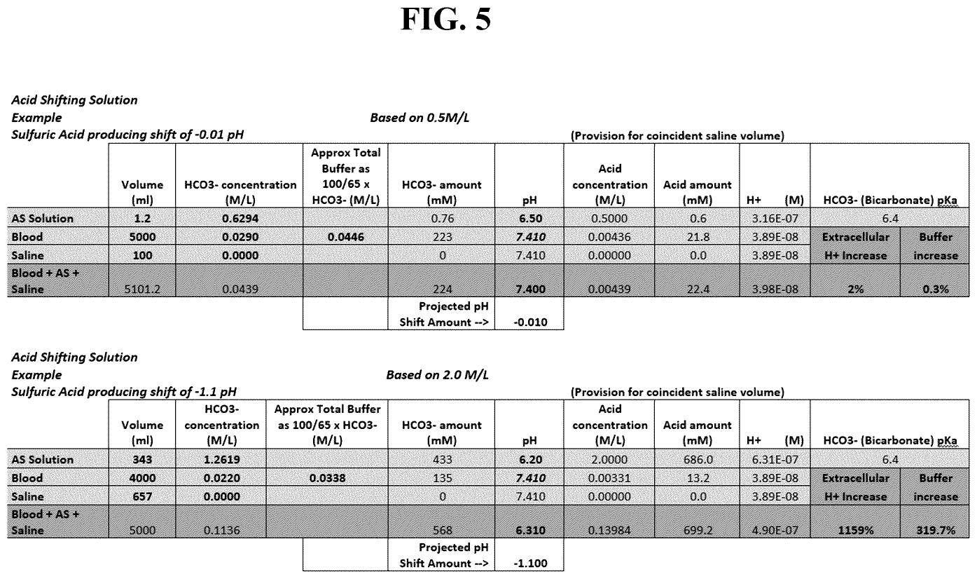

For example, if a small bloodstream shift was desired, an acid shifting solution (AS) may use a 0.5 M sulfuric acid solution. For a patient blood volume of 5 liters, with a high-normal bicarbonate level of 29 mmol/L, and total buffer stores equivalent to 44.6 mM of HCO3−, an infused volume of 1.2 ml containing 0.6 mM of acid would realize a blood acid shift of 0.01 pH. For an infusion procedure to achieve a pH of 6.5, 0.76 mM of a bicarbonate buffer may be used to stabilize the composition prior to administration. This scenario assumes a 100 ml complement of saline also may be administered. Further details of these examples are shown in FIG. 5.

Alternately, if a large bloodstream shift was desired, an acid shifting solution (AS) could be designed utilizing a 2.0 M sulfuric acid solution. If we were to assume that a patient had lost blood volume to retain only 4 liters, had a low-normal bicarbonate level of 22 mmol/L, and contained total buffer stores equivalent to 13.5 mM of HCO3−, an infused volume of 343 ml containing 686 mM of acid would realize a blood acid shift of 1.1 pH. If the infusion procedure desired a pH of 6.2, 43.3 mM of a bicarbonate buffer could be utilized to stabilize the composition prior to administration. This scenario assumes a 657 ml complement of saline might also be administered.

Recognizing that the costs associated with delivering care can be large, one goal may be to improve wellness in as many ways possible during a given intervention, such as an infusion. Further, various ions are commonly administered to address various deficiencies and medical conditions. Accordingly, acid shifting formulations as disclosed herein also may deliver additional benefit through the inclusion of select ions such as, but not limited to, magnesium, potassium, calcium, zinc, copper, selenium, chromium, cobalt, iodine, manganese, and molybdenum. Suh ions are recognized as useful therapeutic choices due to their essential roles in various physiological processes and metabolic functions. Examples of how each of may increase the net benefit if included as a part of an acid-shifting formulation as disclosed herein are provided below, though it will be understood that these ions may be used for other reasons as well.

Magnesium (Mg²⁺): Involved in over 300 enzymatic reactions, including energy production, protein synthesis, and DNA replication. Supports muscle and nerve function, regulates blood pressure, and maintains healthy immune function.

Potassium (K⁺): Crucial for maintaining cellular function, fluid balance, and transmitting nerve impulses. Regulates heart rhythm, supports muscle contraction, and helps prevent hypokalemia (low potassium levels).

Calcium (Ca²⁺): Vital for bone and teeth structure, blood clotting, muscle contraction, and neurotransmitter release. Prevents osteoporosis, supports cardiovascular health, and aids in muscle function.

Zinc (Zn²⁺): Essential for immune function, wound healing, DNA synthesis, and protein production. Supports immune system, promotes skin health, and helps in cell division and growth.

Copper (Cu²⁺): Involved in iron metabolism, formation of red blood cells, and maintenance of nerve cells and the immune system. Supports cardiovascular health, aids in collagen formation, and acts as an antioxidant.

Selenium (Se): Important for antioxidant defense, thyroid hormone metabolism, and immune function. Protects cells from oxidative damage, supports thyroid function, and enhances immune response.

Chromium (Cr³⁺): Enhances insulin action and plays a role in carbohydrate, fat, and protein metabolism. Helps regulate blood sugar levels and supports weight management and metabolism.

Cobalt (Co²⁺): A component of vitamin B12, necessary for red blood cell production and neurological function. Prevents vitamin B12 deficiency, supports nerve function, and aids in DNA synthesis.

Iodine (I⁻): Essential for the synthesis of thyroid hormones, which regulate metabolism, growth, and development. Prevents thyroid disorders such as goiter and hypothyroidism, supports metabolic rate, and promotes healthy growth.

Manganese (Mn²⁺): Involved in bone formation, blood clotting, and antioxidant defense. Supports bone health, helps in the metabolism of amino acids, cholesterol, and carbohydrates, and protects against oxidative stress.

Molybdenum (Mo): Cofactor for enzymes involved in sulfur and amino acid metabolism. Supports detoxification processes, helps in the metabolism of drugs and toxins, and prevents molybdenum deficiency.

Each of these ions contributes to vital biochemical pathways and physiological functions, making their adequate supply essential for maintaining health and preventing deficiencies. Accordingly, infusing these ions may be particularly beneficial in clinical settings where deficiencies or imbalances may occur due to illness, malnutrition, or specific medical conditions, though they may be used in other settings with positive effect as well.

It is also recognized that antioxidants and enzymatic components may be valuable because they play crucial roles in protecting cells from oxidative damage, maintaining redox balance, and supporting overall cellular health. Specifically, tocopherol (aTCP), coenzyme Q10 (Q), taurine, cytochrome c (C) and glutathione (GSH) and enzymatic components including manganese superoxide dismutase (MnSOD), catalase (Cat), glutathione peroxidase (GPX), phospholipid hydroperoxide glutathione peroxidase (PGPX), glutathione reductase (GR); peroxiredoxins (PRX3/5), glutaredoxin (GRX2), thioredoxin (TRX2), and thioredoxin reductase (TRXR2) are recognized as useful therapeutic choices due to their essential roles in various physiological processes and metabolic functions. Examples of how each may contribute to the net benefit if included as a part of an acid-shifting formulation as disclosed herein are provided below, though it will be understood that other benefits may result from use of these components as well, and their use is not limited to these specific examples.

### Antioxidants:

Tocopherol (Vitamin E, aTCP): A lipid-soluble antioxidant that protects cell membranes from oxidative damage by neutralizing free radicals. Reduces the risk of chronic diseases, supports immune function, and promotes skin health.

Coenzyme Q10 (Q): A component of the electron transport chain in mitochondria, crucial for ATP production and as an antioxidant. Supports heart health, enhances energy production, and protects cells from oxidative stress.

Taurine: An amino acid with antioxidant properties, involved in bile salt formation, cell membrane stabilization, and osmoregulation. Protects against cellular damage, supports cardiovascular health, and aids in neurological function.

Cytochrome c (C): A component of the electron transport chain in mitochondria, involved in ATP production and apoptosis regulation. Enhances cellular energy production and helps regulate cell death processes.

Glutathione (GSH): A tripeptide that acts as a major intracellular antioxidant, involved in detoxification and maintenance of redox balance. Protects cells from oxidative damage, supports immune function, and detoxifies harmful substances.

Enzymatic Antioxidants:

Manganese Superoxide Dismutase (MnSOD): An enzyme that converts superoxide radicals into hydrogen peroxide and oxygen, protecting mitochondria from oxidative damage. Reduces oxidative stress, supports mitochondrial function, and prevents cellular damage.

Catalase (Cat): An enzyme that converts hydrogen peroxide into water and oxygen, reducing oxidative stress. Protects cells from hydrogen peroxide toxicity and supports overall cellular health.

Glutathione Peroxidase (GPX): An enzyme that reduces hydrogen peroxide and lipid peroxides using glutathione, protecting cells from oxidative damage. Maintains cellular redox balance and protects against oxidative damage to lipids, proteins, and DNA.

Phospholipid Hydroperoxide Glutathione Peroxidase (PGPX): A specific form of GPX that protects cell membranes from lipid peroxidation. Maintains membrane integrity and prevents oxidative damage to phospholipids.

Glutathione Reductase (GR): An enzyme that regenerates reduced glutathione (GSH) from its oxidized form (GSSG), maintaining glutathione levels in cells. Supports continuous cellular detoxification and antioxidant defense.

Peroxiredoxins (PRX3/5): A family of enzymes that reduce peroxides, protecting cells from oxidative damage. Protects against oxidative stress, supports mitochondrial function, and maintains redox balance.

Glutaredoxin (GRX2): An enzyme that catalyzes the reduction of disulfides in proteins, maintaining redox homeostasis. Protects proteins from oxidative damage and supports cellular redox regulation.

Thioredoxin (TRX2): A protein involved in redox signaling and the reduction of oxidized proteins, maintaining redox homeostasis. Regulates cell growth and apoptosis, and protects against oxidative stress.

Thioredoxin Reductase (TRXR2): An enzyme that regenerates reduced thioredoxin from its oxidized form, supporting cellular redox balance. Maintains cellular redox balance and protects against oxidative stress.

It is also recognized that non-essential amino acids may be valuable additions because they are crucial for protein synthesis, metabolism, immune function, tissue repair and growth, cognitive function, detoxification, and antioxidant defense. Even though the body can synthesize them, there can be a value to supplementing them in different circumstances. Specifically, tyrosine, glycine, arginine, glutamine, glutamic acid, cysteine, serine, proline, alanine, asparagine, and aspartic acid are recognized as interesting therapeutic choices due to their essential roles in various physiological processes and metabolic functions. Examples of how each of these may contribute to the net benefit of an acid-shifting formulation as disclosed herein are provided below, though it will be understood that other benefits may result from use of these components as well, and their use is not limited to these specific examples.
1. Tyrosine: An amino acid involved in the synthesis of neurotransmitters like dopamine, norepinephrine, and epinephrine. Supports cognitive function, mood regulation, and hormone production.
2. Glycine: The simplest amino acid, playing a key role in the synthesis of proteins, collagen, and neurotransmitters. Supports muscle growth, improves sleep quality, and aids in the maintenance of healthy skin and joints.
3. Arginine: An amino acid involved in the production of nitric oxide, which helps relax blood vessels and improve circulation. Supports cardiovascular health, immune function, and wound healing.
4. Glutamine: The most abundant amino acid in the blood, crucial for maintaining the health of the intestinal lining and immune system. Supports gut health, immune function, and muscle recovery.
5. Glutamic Acid: An amino acid that serves as a neurotransmitter in the brain and a precursor to glutamine. Supports cognitive function, energy production, and detoxification processes.
6. Cysteine: A sulfur-containing amino acid involved in the synthesis of glutathione, a major antioxidant. Supports detoxification, immune function, and the health of skin, hair, and nails.
7. Serine: An amino acid involved in the synthesis of proteins, enzymes, and neurotransmitters. Supports cognitive function, immune response, and metabolism of fats and fatty acids.
8. Proline: An amino acid important for the synthesis of collagen, which is vital for the structure of skin, bones, and connective tissues. Supports wound healing, skin health, and joint function.
9. Alanine: An amino acid involved in glucose metabolism and energy production. Supports muscle performance, immune function, and regulation of blood sugar levels.
10. Asparagine: An amino acid involved in the synthesis of proteins and the metabolism of ammonia. Supports nervous system function, protein synthesis, and detoxification processes.
11. Aspartic Acid: An amino acid involved in the citric acid cycle and the synthesis of other amino acids. Supports energy production, neurotransmitter regulation, and liver detoxification.

Routes of administration for a therapeutically effective amount of a composition of the present disclosure include, but are not limited to, intravenous, intramuscular, or parenteral administration, oral administration, optic administration, topical administration, inhalation or otherwise nebulized administration, transmucosal administration and transdermal administration. Compositions of the present disclosure may also be formulated for intravenous, bolus, dermal, oral, optic, suppository, buccal, ocular, or inhalation delivery. For intravenous or parenteral administration, i.e., injection or infusion, the composition may also contain suitable pharmaceutical diluents and carriers, such as water, saline, dextrose solutions, fructose solutions, ethanol, or oils of animal, vegetative, or synthetic origin. It may also contain preservatives, and buffers as are known in the art. When a therapeutically effective amount is administered by intravenous, cutaneous or subcutaneous injection, the solution can also contain components to adjust pH, tonicity, stability, and the like, all of which is within the skill in the art. For topical administration, the composition may be formulated in, e.g., liquid, gel, paste, or cream. In some embodiments, the composition may be administered via a topical patch. For ocular administration, the composition may be formulated in, e.g., liquid eye drops, or as a gel, paste, or cream to be applied to the surface of the eye and/or surrounding tissue. For optic administration, the composition may be formulated in, e.g., ear drops. Other preparations, combinations with other compositions, administration techniques, and the like are described in U.S. Patent Pub. No. 2020/0390743 and U.S. Pat. No. 11,344,529, the disclosures of which are incorporated by reference in their entirety.

The foregoing description, for purpose of explanation, has been described with reference to specific embodiments. However, the illustrative discussions above are not intended to be exhaustive or to limit embodiments of the disclosed subject matter to the precise forms disclosed. Many modifications and variations are possible in view of the above teachings. The embodiments were chosen and described in order to explain the principles of embodiments of the disclosed subject matter and their practical applications, to thereby enable others skilled in the art to utilize those embodiments as well as various embodiments with various modifications as may be suited to the particular use contemplated. Further, specific examples of components that may be used as a part of, or in conjunction with, the compounds and techniques disclosed herein are provided as illustrative examples, and do not limit the scope or content of the embodiments disclosed herein.

The above description includes several example implementations. However, it will be understood by one of skill in the art that the invention disclosed herein is not limited to the implementations described and can be practiced with modification and alteration within the spirit and scope of the appended claims. The description is thus illustrative instead of limiting.

What is claimed is:

1. A method of shifting blood acid for therapeutic purpose while presenting a therapeutic conjugate base, the method comprising:
administering to a subject a therapeutically effective amount of a composition including a buffer solution having at least one pharmaceutical grade acid containing at least one therapeutic conjugate base, and at least one pharmaceutical grade pH buffering agent in a sterile aqueous solution,
wherein the pharmaceutical grade acid comprises an acid selected from a group consisting of Hydroiodic Acid, Perchloric Acid, Chloric Acid, Sulfuric Acid, Nitric Acid, Folic Acid, Sulfurous Acid, Sulfuric Acid, Glutamic Acid, and Pyruvic Acid, or a combination of two or more thereof,
wherein the therapeutic conjugate base comprises a base selected from a group consisting of Iodine, Perchlorate, Chlorate, Sulfate, Nitrate, Folate, Bisulfite, Glutamate, and Pyruvate, or a combination of two or more thereof, or ionized or radioactive forms thereof,
wherein the concentration of the pharmaceutical grade acid and the pharmaceutical grade pH buffering agent in the buffer solution is sufficient to provide a total acid content of from 60 mmol/L to 3,000 mmol/L when administered to a subject, and
wherein the selected pharmaceutical grade acid and the pharmaceutical grade pH buffering agent or agents provide a buffer solution pH of between 1.8 and 8.6 when administered to a subject.

2. The method of claim 1, wherein the buffer solution is administered in an amount that is sufficient to reduce the physiological bloodstream pH of a subject by 0.01 to 1.1.

3. The method of claim 1, wherein the buffer solution is administered in an amount sufficient to reduce the physiological bloodstream pH of a subject by 0.15 to 0.75.

4. The method of claim 1, wherein the concentration of the acid and buffering agent provides the buffer solution with a buffer capacity sufficient to sustain the reduction of the physiological bloodstream pH of the subject for between 1 minute and 1 week.

5. The method of claim 1, wherein the therapeutic intent is treatment of diabetes, insulin resistance, glucose intolerance, hyperglycemia, hyperinsulinemia, obesity, hyperlipidemia, hyperlipoproteinemia, cancer, sepsis, trauma care, treating wounds, reducing vascular plaque, treating radiation exposure, enhancing glutathione status, enhancing sulfur-based metabolism, and/or infectious disease.

6. The method of claim 1, wherein the composition further comprises one or more ion sources selected from a group consisting of: a magnesium ion source, a potassium ion source, a calcium ion source, a zinc ion source, a copper ion source, a selenium ion source, a chromium ion source, a cobalt ion source, an iodine ion source, a manganese ion source, and a molybdenum ion source.

7. The method of claim 1, wherein the composition further comprises one or more vitamins selected from a group consisting of: a B vitamin, vitamin C, and vitamin K.

8. The method of claim 1, wherein the composition further comprises antioxidant defense compounds comprising one or more nonenzymatic compounds selected from the group consisting of: tocopherol (aTCP), coenzyme Q10 (Q), taurine, cytochrome c (C) and glutathione (GSH) and enzymatic components including manganese superoxide dismutase (MnSOD), catalase (Cat), glutathione peroxidase (GPX), phospholipid hydroperoxide glutathione peroxidase (PGPX), glutathione reductase (GR); peroxiredoxins (PRX3/5), glutaredoxin (GRX2), thioredoxin (TRX2), thioredoxin reductase (TRXR2), and a combination of two or more thereof.

9. The method of claim 1, wherein the composition further comprises one or more essential amino acids selected from a group consisting of: histidine, isoleucine, leucine, lysine, methionine, phenylalanine, threonine, tryptophan, valine and a combination of two or more thereof.

10. The method of claim 1, wherein the composition further comprises one or more nonessential amino acids selected from a group consisting of: tyrosine, glycine, arginine, glutamine, glutamic acid, cysteine, serine, proline, alanine, asparagine, aspartic acid, and a combination of two or more thereof.

11. The method of claim 1, wherein the composition is formulated in hypotonic, isotonic, or hypertonic form.

12. The method of claim 1, wherein the composition is administered intravenously, by bolus, dermally, orally, optic, via suppository, buccally, or via inhalation.

13. The method of claim 1, wherein the administering comprises introducing the composition by infusion over a period of about 1 minute to about 1 hour, and the infusion is repeated as necessary over a period of time selected from about 1 day to about 1 year.

14. The method of claim 1, wherein one or more alkaline-shifting solutions are employed before or after one or more acid-shifting solutions.

15. The method of claim 1, wherein one or more alkaline-shifting solutions are alternatively administered between one or more acid shifting solutions.

16. The method of claim 1, wherein one or more acid-shifting solutions are alternatively administered between one or more alkaline-shifting solutions.

17. The method of claim 1, wherein the subject is a human or veterinary subject or culture thereof.

18. A pharmaceutical composition for intravenous delivery to a mammal, the pharmaceutical composition comprising:
an intravenous buffer solution comprising at least one pharmaceutical grade acid containing at least one therapeutic conjugate base; and
at least one pharmaceutical grade pH buffering agent in a sterile aqueous solution.

19. The pharmaceutical composition of claim 18, wherein the pharmaceutical grade acid comprises an acid selected from a group consisting of Hydroiodic Acid, Perchloric Acid, Chloric Acid, Sulfuric Acid, Nitric Acid, Folic Acid, Sulfurous Acid, Sulfuric Acid, Glutamic Acid, and Pyruvic Acid, or a combination of two or more thereof.

20. The pharmaceutical composition of claim 18, wherein the therapeutic conjugate base comprises a base selected from a group consisting of Iodine, Perchlorate, Chlorate, Sulfate, Nitrate, Folate, Bisulfite, Glutamate, and Pyruvate, or a combination of two or more thereof, or ionized or radioactive forms thereof.

21. The pharmaceutical composition of claim 18, wherein the concentration of the pharmaceutical grade acid and the pharmaceutical grade pH buffering agent in the buffer solution is sufficient to provide a total acid content of from 60 mmol/L to 3,000 mmol/L when administered; and wherein the selected pharmaceutical grade acid and the pharmaceutical grade pH buffering agent or agents provide a buffer solution pH of between 1.8 and 8.6 when administered.
